(19) United States
(12) Patent Application Publication
(10) Pub. No.: US 2023/0416181 A1
(43) Pub. Date: Dec. 28, 2023

(54) OXYGENATED SOLVENT ODORANT REMOVAL COMPOSITION

(71) Applicant: Dow Global Technologies LLC, Midland, MI (US)

(72) Inventors: Cheng Shen, Shanghai (CN); Jian Zou, Shanghai (CN); Shuyu Duan, Shanghai (CN); Hongying Wang, Shanghai (CN); Jing Ji, Shanghai (CN)

(21) Appl. No.: 18/255,719

(22) PCT Filed: Dec. 18, 2020
(86) PCT No.: PCT/CN2020/137609

§ 371 Date: Jun. 2, 2023

(51) Int. Cl. C07C 41/46

(52) U.S. Cl. CPC C07C 41/46

(57) ABSTRACT

An odor removal composition for oxygenated solvents comprising an alcohol amine.

### FIELD

Embodiments of the present disclosure generally relate to odorant removers and methods of controlling odor in oxygenated solvents, wherein the odorant remover comprises at least an amino alcohol.

### INTRODUCTION

Consumers have increasing awareness and concerns on indoor and outdoor air quality. Even though more and more coating formulations have switched to a water-based formulation, there is a clear market need driving coating producers to deliver coatings with less VOC (volatile organic compounds) emissions and lower odor. More restrictive regulations around VOC emissions have also increased the demand for lower odor coatings.

Dipropylene glycol mono butyl ether (DPnB) and dipropylene glycol mono methyl ether (DPM) are two examples of commonly used oxygenated solvents in water-based wood coating. There is a high demand for these glycol ether solvents to have lower odor. However, DPnB and DPM at present often contain some impurities in the final compositions. These impurities may contribute to high VOC emission and induce a strong odor during evaporation of the solvent (s). Additionally, the amount of these impurities may further increase under heating conditions due to oxidation, which can contribute to higher VOC emissions and stronger odor.

Generally, impurities in oxygenated solvent products include aldehydes, ketones, acids, esters, and their derivatives, etc. These impurities could be introduced from the alcohols (used as raw materials in solvent production), generated during alkoxylation process, or formed by oxidation during storage. This oxidation may be accelerated at higher temperatures which leads to a break-down of the alkoxylate chain with formaldehyde (a VOC) formed as one of many degradation products.

For all these reasons and more, there is a need for an odor control package and method of controlling odor in oxygenated solvents.

### SUMMARY

Embodiments of the present invention generally relate to an amino alcohol odorant remover which, along with an antioxidant effectively reduces the odorant contents in oxygenated solvents. Embodiments of the present invention also relate to methods of controlling odor in oxygenated solvents by use of an odor removal composition. Embodiments of the present invention also relate to oxygenated solvents comprising an odor removal composition.

### DETAILED DESCRIPTION

The present disclosure relates to an odorant remover and method of controlling odors and volatile chemical compounds (VOCs) in oxygenated solvents. In one embodiment, the odorant remover may be an amino alcohol which blends with an antioxidant to effectively reduce the odorant contents in oxygenated solvents. This odorant remover acts to reduce odorants at lower dosages and can enable easy processing conditions with improved performance.

The odor removal composition comprises an amino alcohol. The general structure of the amino alcohol used in the odor removal composition, in one embodiment, is shown below:

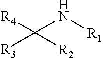

wherein, R₁, R₂ and R₃ may be a H, alkylamine, or hydroxyl alkyl group with a linear or branched carbon chain ranging from C1-C8. R₄ may be an alkylamine or hydroxyl alkyl group with linear or branched carbon chain ranging from C1-C8. Examples of amino alcohols that can be used, in some embodiments, include, but are not limited to diethanolamine (DEA, CAS #: 111-42.2) or aminoethyl ethanolamine (AEEA, CAS #: 111-41-1).

The odor removal composition may also comprise at least one antioxidant. The antioxidants may include, but are not limited to, phenolic anti-oxidants such as synthetic Vitamin E (d,l-a-tocopherol, CAS #: 10191-41-0) and propyl gallate (CAS #: 121-79-9).

The reduction of odors in oxygenated solvents may be achieved by a number of different methods. For example, in one embodiment, the odorant remover(s) and antioxidant(s) may be added directly into the oxygenated solvents after the solvents are produced. This is remarkable and advantageous because post-process addition of the odorant remover(s) means there is little impact upon the current solvent manufacturing processes. This provides a cost effective, low impact means to achieve lower odor oxygenated solvents.

In one embodiment, a method of controlling odor in oxygenated solvents comprises (a) providing an oxygenated solvent having the following formula: R₁—O—(CHR₂CHR₃)O)nR₄, wherein R₁ ranges from C1-C9 linear or branched alkyl group or is a phenyl group, R₂ and R₃ are H or C1-C2 alkyl, wherein R₂ is H when R₃ is C1-C2, and R₃ is H when R₂ is C1-C2, and R₄ is H and n is an integer from 1-6; and (b) adding at least one alcohol amine to the oxygenated solvent, the at least one alcohol amine having the following structure:

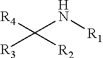

wherein, R₁, R₂ and R₃ are H, alkylamine, or hydroxyl alkyl group with linear or branched carbon chain ranging from C1-C8, and R₄ is an alkylamine or hydroxyl alkyl group with linear or branched carbon chain ranging from C1-C8. In some embodiments, the method further comprises adding at least one antioxidant to the oxygenated solvent. The antioxidant may include, but is not limited to, phenolic anti-oxidants such as synthetic Vitamin E (d,l-a-tocopherol, CAS #: 10191-41-0) and propyl gallate (CAS #: 121-79-9).

In one embodiment, an odor removal composition comprises 0.001 to 1 weight percent of an amino alcohol as described herein, 0 to 1 weight percent of at least one phenolic antioxidant, less than 1 weight percent water, and greater 90 weight percent of an oxygenated solvent, each based on the total weight of the composition. In another preferred embodiment, an odor removal composition comprises 0.001 to 0.25 weight percent of an amino alcohol as described herein, 0 to 0.25 weight percent of at least one phenolic antioxidant, less than 0.5 weight percent water, and greater 95 weight percent of an oxygenated solvent, each based on the total weight of the composition. In yet another preferred embodiment, an odor removal composition comprises 0.001 to 0.1 weight percent of an amino alcohol as described herein, 0.001 to 0.1 weight percent of at least one phenolic antioxidant, less than 0.3 weight percent water, and greater 98 weight percent of an oxygenated solvent, each based on the total weight of the composition.

The oxygenated solvents may include but are not limited to solvents which have the following formula: R₁—O—(CHR₂CHR₃)O)nR₄, wherein R₁ ranges from C1-C9 linear or branched alkyl group or is a phenyl group, R₂ and R₃ are H or C1-C2 alkyl, wherein R₂ is H when R₃ is C1-C2, and R₃ is H when R₂ is C1-C2, and R₄ is H and n is an integer from 1-6.

Yet other examples of oxygenated solvents may include but are not limited to dipropylene glycol mono butyl ether and dipropylene glycol mono methyl ether (DOWANOL™ DPM Glycol Ether and DOWANOL™ DPnB Glycol Ether available from Dow Chemical) and butanol initiated ethoxylated solvents (Butyl CARBOTOL™ available from Dow Chemical).

The present invention may be utilized to remove odors from newly produced batches of oxygenated solvents and used to treat oxygenated solvents stored for long periods. Stored oxygenated solvents tend to produce more odor molecules such as cyclic ethers 2,4-dimethyl-1,3-dioxolane (DMD), 2-ethyl-4-methyl-1,3-dioxolane (EMD) or trioxocane, that are themselves strong odorants. DMD and EMD may be formed by derivation of propylene glycol and acetaldehyde or propionaldehyde during the storage. The present invention can be added to an oxygenated solvent and mitigate these odorants for a long time period.

In another embodiment of the present invention, the amino alcohol shown below may be combined with an antioxidant without the need of an oxygenated solvent being present:

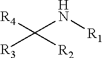

wherein, R₁, R₂ and R₃ may be a H, alkylamine, or hydroxyl alkyl group with a linear or branched carbon chain ranging from C1-C8. R₄ may be an alkylamine or hydroxyl alkyl group with linear or branched carbon chain ranging from C1-C8.

Some embodiments of the present invention also relate to oxygenated solvents comprising an odor removal composition. In some embodiments, such oxygenated solvents comprise: an oxygenated solvent having the following formula: R₁—O—(CHR₂CHR₃)O)nR₄, wherein R₁ ranges from C1-C9 linear or branched alkyl group or is a phenyl group, R₂ and R₃ are H or C1-C2 alkyl, wherein R₂ is H when R₃ is C1-C2, and R₃ is H when R₂ is C1-C2, and R₄ is H and n is an integer from 1-6; and
(a) at least one alcohol amine with the structure of:

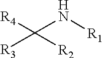

wherein R₁, R₂ and R₃ are H, alkylamine, or hydroxyl alkyl group with linear or branched carbon chain ranging from C1-C8, and R₄ is an alkylamine or hydroxyl alkyl group with linear or branched carbon chain ranging from C1-C8. In some embodiments, the oxygenated solvent further comprises at least one antioxidant. The antioxidants may include, but are not limited to, phenolic anti-oxidants such as synthetic Vitamin E (d,l-a-tocopherol, CAS #: 10191-41-0) and propyl gallate (CAS #: 121-79-9). In some embodiments, the oxygenated solvent comprises 0.001 to 1 weight percent of the amino alcohol, 0 to 1 weight percent of at least one phenolic antioxidant, less than 1 weight percent water, and greater 90 weight percent of the oxygenated solvent, each based on the total weight of the composition. In another embodiment, the oxygenated solvent comprises 0.001 to 0.25 weight percent of the amino alcohol as described herein, 0 to 0.25 weight percent of at least one phenolic antioxidant, less than 0.5 weight percent water, and greater 95 weight percent of the oxygenated solvent, each based on the total weight of the composition. In yet another embodiment, the oxygenated solvent comprises 0.001 to 0.1 weight percent of the amino alcohol, 0.001 to 0.1 weight percent of at least one phenolic antioxidant, less than 0.3 weight percent water, and greater 98 weight percent of the oxygenated solvent, each based on the total weight of the composition.

Some embodiments of the present invention will now be discussed in the following Examples.

### EXAMPLES

The following Examples test the efficacy of the presently disclosed odor removal compositions and others.

### I. Materials

TABLE 1
Raw Materials

| Additive | CAS # | Structure | Description | Supplier |
|---|---|---|---|---|
| Aminoethyl ethanolamine (AEEA) | 111-41-1 | 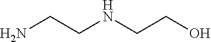 | Amino alcohol odorant remover | Dow Chemical |
| Diethanolamine (DEA) | 111-42-2 | 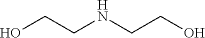 | Amino alcohol odorant remover | Dow Chemical |
| Tris-(hydroxyl- methyl) amino- methane (Tris- Amine) | 77-86-1 | 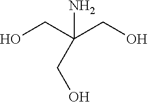 | Amino alcohol odorant remover | Sino- Pharma |
| Synthetic Vitamin E (DL- α-Tocopherol) (SVE) | 10191- 41-0 | 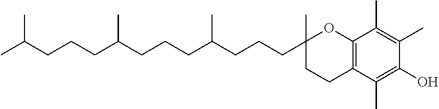 [SMILES: Cc1c(C)c2c(c(C)c1O)CCC(C)(CCCC(C)CCCC(C)CCCC(C)C)O2] | antioxidant | Sigma- Aldrich |
| Propyl gallate (PG) | 121-79-9 | 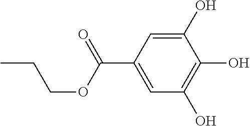 | antioxidant | Sino- Pharma |
| DOWANOL ™ | 29911-28-2 | Dipropylene glycol mono butyl ether | Oxygenated | Dow |
| DPnB Glycol | | | solvent | Chemical |
| Ether | | | | |
| DOWANOL ™ | 34590-94-8 | Dipropylene glycol mono methyl ether | Oxygenated | Dow |
| DPM Glycol | | | solvent | Chemical |
| Ether | | | | |
| Butyl | 112-34-5 | Diethylene glycol mono butyl ether | Oxygenated | Dow |
| CARBOTOL ™ | | | solvent | Chemical |

### II. Odor Removal Tests

Testing Methodology

A certain amount of amino alcohol and/or antioxidant is added into an amount of a given solvent (around 20 mL) at room temperature (see Table 2 for all tested formulations). Examples are denoted by the suffix “IE” which stands for inventive example (e.g., IE-M7) in the test results below. Other comparative examples containing no amino alcohol and/or antioxidant (or either) are also prepared for each round of testing and are denoted with “CE” suffix (e.g., CE-M1) in the results below.

The mixtures of amino alcohol and/or antioxidant and solvent (or comparatives) are then kept on a shaking table for 2 h at 300 RPM to obtain a homogeneous appearance. Once this shaking is completed, the mixture is then kept at room temperature for 48 hours, then subjected to various forms of odorant testing including: Headspace GC-MS analysis, the SPME PFBHA derivatization GC-MS method, and the SPME GC-MS method. The details of each of these testing methodologies are listed below. Results for this portion of the testing may be found in Tables 3-4. The dosage of odorant remover(s) may also be optimized by combining amino alcohol and antioxidant. Results for this portion of the testing may be found in Tables 5-6. The odorant removal capabilities upon aged oxygenated solvents may also be tested, with results for this portion of the testing found in Tables 7-8. The odorant removal capabilities upon EO based oxygenated solvents may also be tested, with results for this portion of the testing found in Table 9.

Headspace GC-MS Method:

Headspace GC-MS Instrument: 7890A Gas Chromatograph, 5975C Mass Spectrometer with a 7697A headspace auto sampler. GC column: SOLGEL-wax (sn. 1297586B08, p/n 054787), 30 m×250 μm×0.25 μm. Carrier gas: helium carrier gas at 1.0 mL/min constant flow. GC oven program: 50° C. holding for 5 min, 10° C./min ramp to 250° C., holding for 3 min. MSD parameters (scan mode): MS source temperature: 230° C., MS Quad temperature: 150° C., Acq. Mode: Scan, Mass from 29 to 400 Da. Headspace oven condition: heated at 130° C. for 15 min. Sample preparation: 20-30 mg of sample was put into a 20-mL headspace vial for analysis. Several samples in one test were prepared for triplicate, and the average results were reported. All VOCs were semi-quantified using toluene as standard. An aliquot of 4 μL toluene solution (500 μg/g, prepared in acetonitrile (ACN)) was injected into headspace vial, and toluene peak area was used for semi-quantification.

SPME On-Fiber Derivatization Method:

The analysis of acetaldehyde presence was conducted using SPME on-fiber derivatization method. The SPME on-fiber derivatization method parameters were as following: Headspace GC-MS Instrument: 7890A Gas Chromatograph, 5975C Mass Spectrometer with a 7697A headspace auto sampler. GC column: DB-5, 30 m×250 μm×μm. Carrier gas: helium carrier gas at 1.0 mL/min constant flow. MSD parameters (scan mode): MS source temperature: 230° C., MS Quad temperature: 150° C., Acq. Mode: Scan, Mass from 29 to 400 Da. Oven program: 50° C. for 3 min, and then 15° C./min to 180° C. for 0 min. Sample preparation: 0.5 g of sample was added into 20 mL headspace vial. Aldehyde standards: 2 μL of mixture of each aldehyde (1 ppm of each) was injected into 20 mL headspace vial for quantification of various aldehydes.

SPME on-fiber derivatization parameters were as below:
SPME fiber type: 65 μm PDMS-DVB (Supleco. Co. ltd, 57321-U)
On fiber derivatization: 5 min, 50° C.
Derivatization agent: 0-(2,3,4,5,6-Pentafluorobenzyl) hydroxylamine hydrochloride (PFBHA·HCl, 99+%). 1 mL in 20 mL headspace vial (17 mg/mL).
Incubation time: 5 min, 60° C.
Extraction: 5 min, 60° C.

SPME (Solid Phase Micro-Extraction) GC-MS Method:

SPME GC-MS analysis was conducted on an Agilent 6890 gas chromatograph coupled with a mass spectrometry detector (Agilent 5975C MSD). The GC conditions are listed below. Semi-quantification was conducted by a reference standard (5 ppm of each, prepared in polyol 8010).

Oven program: Initial temp: 50° C. (On), Maximum temp: 325° C., Initial time: 4.00 min, equilibration time: 0.50 min. Ramps: Rate (16) Final (250) temp (2) Run time: 18.50 min Ambient temp: 25° C. SPME condition: PDMS/DVB SPME fiber from Supleco Co. ltd, Incubation Temp.: 75° C., Incubation Time: 5.00 min, Extraction Time: 30 min

Agilent 19091S-433, HP-5MS, 5% Phenyl Methyl Silox, 30 m×250 μm, 0.25 μm film thickness, Mode: constant pressure, Pressure: 7.6522 psi, Nominal initial flow: 1 mL/min, Average velocity: 36.445 cm/sec
MS SCAN and SIM parameters: DMD/EMD quantification: Resolution: Low Group Start Time: 2.30, Plot 1 Ion: 72.00 Ions/Dwell in Group (Mass, Dwell) (Mass, Dwell) (Mass, Dwell) (59.00, 30) (72.00, 30) (87.00, 30). Trioxocane quantification: Resolution: Low, Group Start Time: 8.00, Plot 1 Ion: 101.00, Ions/Dwell In Group (Mass, Dwell) (Mass, Dwell) (Mass, Dwell) (59.00, 30) (101.00, 30) (130.00, 30)

TABLE 2
Odor Removal Test Formulations

| Examples | Alcohol Amine | Amount | Antioxidant | Amount | Solvent |
|---|---|---|---|---|---|
| CE-M1 | N/A | N/A | N/A | N/A | DPM |
| IE-M2 | Tris-amine | 500 PPM | N/A | N/A | DPM |
| IE-M3 | DEA | 500 PPM | N/A | N/A | DPM |
| IE-M4 | AEEA | 500 PPM | N/A | N/A | DPM |
| CE-B1 | N/A | N/A | N/A | N/A | DPnB |
| IE-B2 | Tris-amine | 500 PPM | N/A | N/A | DPnB |
| IE-B3 | DEA | 500 PPM | N/A | N/A | DPnB |
| IE-B4 | AEEA | 500 PPM | N/A | N/A | DPnB |
| CE-M5 | N/A | N/A | N/A | N/A | DPM |
| CE-M6 | N/A | N/A | SVE | 100 PPM | DPM |
| CE-M7 | N/A | N/A | PG | 100 PPM | DPM |
| IE-M8 | AEEA | 100 PPM | N/A | N/A | DPM |
| IE-M9 | DEA | 100 PPM | N/A | N/A | DPM |
| IE-M10 | AEEA | 100 PPM | SVE | 100 PPM | DPM |
| IE-M11 | DEA | 100 PPM | SVE | 100 PPM | DPM |
| CE-B5 | N/A | N/A | N/A | N/A | DPnB |
| CE-B6 | N/A | N/A | SVE | 100 PPM | DPnB |
| CE-B7 | N/A | N/A | PG | 100 PPM | DPnB |
| IE-B8 | AEEA | 100 PPM | N/A | N/A | DPnB |
| IE-B9 | DEA | 100 PPM | N/A | N/A | DPnB |
| IE-B10 | AEEA | 100 PPM | SVE | 100 PPM | DPnB |
| IE-B11 | DEA | 100 PPM | SVE | 100 PPM | DPnB |
| CE-M12 | N/A | N/A | N/A | N/A | DPM |
| CE-M13 | N/A | N/A | N/A | N/A | DPM |
| IE-M14 | AEEA | 100 PPM | N/A | N/A | DPM |
| CE-M15 | N/A | N/A | SVE | 100 PPM | DPM |
| IE-M16 | AEEA | 100 PPM | SVE | 100 PPM | DPM |
| CE-B12 | N/A | N/A | N/A | N/A | DPnB |
| CE-B13 | N/A | N/A | N/A | N/A | DPnB |
| IE-B14 | AEEA | 100 PPM | N/A | N/A | DPnB |
| CE-B15 | N/A | N/A | SVE | 100 PPM | DPnB |
| IE-B16 | AEEA | 100 PPM | SVE | 100 PPM | DPnB |
| CE-BC1 | N/A | N/A | N/A | N/A | Buty1 |
| | | | | | CARBOTOL ™ |
| IE-BC2 | AEEA | 100 PPM | N/A | N/A | Butyl |
| | | | | | CARBOTOL ™ |
| CE-BC3 | N/A | N/A | SVE | 100 PPM | Buty1 |
| | | | | | CARBOTOL ™ |
| IE-BC4 | AEEA | 100 PPM | SVE | 100 PPM | Butyl |
| | | | | | CARBOTOL ™ |

Results

TABLE 3
Results of odorant removal for DOWANOL ™ DPM

| | | Examples | | | |
|---|---|---|---|---|---|
| | | | IE-M2 | IE-M3 | IE-M4 |
| | R.T. | CE-M1 | Tris-amine | DEA | AEEA |
| Odorants | (min) | Ref Av. | 500 ppm | 500 ppm | 500 ppm |
| Acetaldehyde | 1.44 | 77.6 | 33.6 | <LOQ | <LOQ |
| Methyl formate | 1.59 | 14.8 | 16.3 | 5.7 | 1.8 |
| Acetic acid, methyl ester | 1.72 | 59.4 | 50.3 | 10.5 | 4.8 |
| Methyl alcohol | 1.94 | 3.9 | 2.5 | <LOQ | <LOQ |
| 1,3-Dioxane, 2-methyl- or isomer | 2.13 | 20.6 | 22.4 | 3.6 | <LOQ |
| 2-Propanone, 1-methoxy- | 4.00 | 19.1 | 13.6 | 2.5 | 0.3 |
| 2-Propanol, 1-methoxy- | 4.71 | 2.7 | 7.9 | 4.8 | 6.9 |
| Propane, 1,3-dimethoxy- | 6.50 | 34.4 | 28.7 | 5.6 | 4.3 |
| Propionic acid, 2-isopropoxy-, | 7.00 | 38.7 | 29.2 | 4.2 | 2.4 |
| methyl ester | | | | | |
| Ethanol, 2-methoxy-, acetate | 7.86 | 1.6 | 1.7 | 0.1 | <LOQ |
| Lactic acid | 8.30 | 1.2 | 0.4 | <LOQ | <LOQ |
| 2-Propanone, 1-hydroxy- | 8.60 | 9.8 | 11.3 | 2.1 | 44.5 |
| Trioxocane | <LOQ | <LOQ | <LOQ | <LOQ | <LOQ |
| Sum (ppm) | | 283.8 | 217.9 | 39.1 | 65.0 |
| Removal ratio (%) | | | 23.22 | 86.22 | 77.10 |

Note:
units are in PPM ((by way of the headspace GC-MS method) and LOQ (limit of quantification) is around 0.1 ppm (very low level).

TABLE 4
Results of odorant removal for DOWANOL ™ DPnB

| | | Examples | | | |
|---|---|---|---|---|---|
| | | | IE-B2 | IE-B3 | IE-B4 |
| | R.T. | CE-B1 | Tris-amine | DEA | AEEA |
| Odorant | (min) | Ref Av. | 500 ppm | 500 ppm | 500 ppm |
| Butanal | 1.88 | 296.3 | 74.8 | 12.2 | 8.0 |
| Formic acid, butyl ester | 2.95 | 439.4 | 94.9 | 18.5 | 6.6 |
| Acetic acid, butyl ester | 8.81 | 33.1 | 5.8 | 0.8 | <LOQ |
| 2-Propanone, 1-hydroxy- | 9.33 | 30.7 | 9.0 | <LOQ | <LOQ |
| Propanoic acid, 2,2-dimethyl- | 10.71 | 165.3 | 39.0 | 4.1 | 2.5 |
| Acetic acid | 11.42 | 132.1 | 14.3 | <LOQ | <LOQ |
| 2-Propanone, 1-(acetyloxy)- | 11.64 | 85.3 | 20.7 | 4.0 | 0.8 |
| 1,2-Propanediol, 1-acetate | 13.17 | 88.1 | 15.6 | 1.6 | 0.5 |
| Sum (ppm) | | 1270.3 | 274.1 | 41.2 | 18.4 |
| Removal ratio (%) | | | 78.42 | 96.76 | 98.55 |

Note:
units are in PPM ((by way of the headspace GC-MS method) and LOQ is around 0.1 ppm (very low level).

TABLE 5
Dosage optimization of odorant removers in DOWANOL ™ DPM

| | | Examples | | | | | | |
|---|---|---|---|---|---|---|---|---|
| | | CE-M5 | CE-M6 | CE-M7 | IE-M8 | IE-M9 | IE-M10 | IE-M11 |
| Odorants | R.T. | Ref | SVE | PG | AEEA | DEA | AEEA + SVE | DEA + SVE |
| | (min) | Av. | 100 ppm | 100 ppm | 100 ppm | 100 ppm | 100 + 100 | 100 + 100 |
| | | | | | | | ppm | ppm |
| Acetaldehyde | 1.44 | 77.6 | 55.9 | 9.0 | 2.6 | 19.6 | 2.3 | 19.7 |
| Methyl formate | 1.59 | 14.8 | 10.5 | 5.3 | 2.6 | 7.3 | 2.5 | 2.8 |
| Acetic acid, methyl ester | 1.72 | 59.4 | 37.2 | 9.5 | 9.8 | 27.4 | 8.5 | 11.5 |
| Methyl alcohol | 1.94 | 3.9 | 3.2 | 2.1 | 2.7 | 2.8 | 2.3 | 3.4 |
| 1,3-Dioxane, 2-methyl- | 2.13 | 20.6 | 15.3 | 5.3 | <LOQ | 11.3 | 3.1 | 4.9 |
| or isomer | | | | | | | | |
| 2-Propanone, 1-methoxy- | 4.00 | 19.1 | 4.8 | 0 | 3.3 | 5.4 | 2.5 | 3.0 |
| 2-Propanol, 1-methoxy- | 4.71 | 2.7 | 3.0 | 7 | 4.5 | 4.3 | 3.9 | 3.7 |
| Propane, 1,3-dimethoxy- | 6.50 | 34.4 | 20.8 | 6.6 | 5.5 | 13.5 | 5.5 | 7.8 |
| Propionic acid, 2-isopropoxy-, | 7.00 | 38.7 | 20.3 | 1.5 | 4.2 | 16.0 | 5.2 | 6.8 |
| methyl ester | | | | | | | | |
| Ethanol, 2-methoxy-, | 7.86 | 1.6 | <LOQ | 0.8 | <LOQ | <LOQ | <LOQ | 0.2 |
| acetate | | | | | | | | |
| Lactic acid | 8.30 | 1.2 | <LOQ | 0 | <LOQ | <LOQ | <LOQ | <LOQ |
| 2-Propanone, 1-hydroxy- | 8.60 | 9.8 | 4.1 | 1.5 | 1.9 | 1.6 | 0.9 | 1.9 |
| Trioxocane | | <LOQ | <LOQ | 0.9 | <LOQ | <LOQ | <LOQ | <LOQ |
| Sum (ppm) | | 283.8 | 175.1 | 49.5 | 37.1 | 109.2 | 36.7 | 65.7 |
| Removal ratio (%) | | | 38.30 | 83.05 | 86.93 | 61.52 | 87.07 | 76.85 |

Note:
units are in PPM ((by way of the headspace GC-MS method) and LOQ is around 0.1 ppm (very low level).

TABLE 6
Dosage optimization of odorant removers in DOWANOL ™ DPnB

| | | Examples | | | | | | |
|---|---|---|---|---|---|---|---|---|
| | | CE-B5 | CE-B6 | CE-B7 | IE-B8 | IE-B9 | IE-B10 | IE-B11 |
| Odorant | R.T. | Ref | SVE | PG | AEEA | DEA | AEEA + SVE | DEA + SVE |
| | (min) | Av. | 100 ppm | 100 ppm | 100 ppm | 100 ppm | 100 + 100 | 100 + 100 |
| | | | | | | | ppm | ppm |
| Butanal | 1.88 | 296.3 | 28.6 | 50.7 | 20.1 | 53.5 | 16.9 | 22.8 |
| Formic acid, butyl ester | 2.95 | 439.4 | 52.3 | 46.4 | 21.7 | 60.7 | 22.2 | 29.7 |
| Acetic acid, butyl ester | 8.81 | 33.1 | 3.8 | 7.0 | 1.4 | 4.1 | 1.7 | 1.7 |
| 2-Propanone, 1-hydroxy- | 9.33 | 30.7 | <LOQ | 9.3 | <LOQ | <LOQ | <LOQ | <LOQ |
| Propanoic acid, 2,2-dimethyl- | 10.71 | 165.3 | 10.6 | 45.8 | 5.6 | 21.1 | 4.6 | 7.9 |
| Acetic acid | 11.42 | 132.1 | 8.3 | 6.5 | 1.5 | 7.4 | 0.9 | 2.7 |
| 2-Propanone, 1-(acetyloxy)- | 11.64 | 85.3 | 16.3 | 19.0 | 3.3 | 14 | 3.4 | 7.7 |
| 1,2-Propanediol, 1-acetate | 13.17 | 88.1 | 9.7 | 18.9 | 0.6 | 6.7 | 1.1 | 3.5 |
| Sum (ppm) | | 1270.3 | 129.6 | 203.6 | 54.2 | 167.5 | 50.8 | 76.0 |
| Removal ratio (%) | | | 89.80 | 83.97 | 95.73 | 86.81 | 96.00 | 94.02 |

Note:
units are in PPM ((by way of the headspace GC-MS method) and LOQ is around 0.1 ppm (very low level).

TABLE 7
Odorant results of DPM after 3-month heat-ageing at 54° C.

| | Examples | | | | |
|---|---|---|---|---|---|
| | | | | | IE-M16 |
| | CE-M12 | CE-M13 | IE-M14 | CE-M15 | AEEA + SVE |
| | Ref. | Ref. | AEEA | SVE | 100 ppm + |
| Additive | Fresh | Aged | 100 ppm | 100 ppm | 100 ppm |
| Acetaldehyde | 77.6 | 236.5 | 56.9 | 57.2 | 35.2 |
| Methyl formate | 14.8 | 124.6 | 15.4 | 27.5 | 11.2 |
| Acetic acid, methyl ester | 59.4 | 489.1 | 71.5 | 95.9 | 55 |
| Methyl alcohol | 3.9 | 71.7 | 14.2 | 9.7 | 5.7 |
| 1,3-Dioxane, 2-methyl- or isomer | 20.6 | 221.2 | 46.4 | 77 | 31.9 |
| 2-Propanone, 1-methoxy- | 19.1 | 231.0 | 21.8 | 35.1 | 15.9 |
| 2-Propanol, 1-methoxy- | 2.7 | 265.6 | 14.5 | 11.5 | 16.5 |
| Propane, 1,3-dimethoxy- | 34.4 | 444.4 | 47.2 | 66 | 36. |
| Propionic acid, 2-isopropoxy-, | 38.7 | 396.7 | 38.1 | 57.4 | 27.2 |
| methyl ester | | | | | |
| Ethanol, 2-methoxy-, acetate | 1.6 | 20.5 | <LOQ | <LOQ | <LOQ |
| Lactic acid | 1.2 | 8.9 | 3.7 | <LOQ | 1.5 |
| 2-Propanone, 1-hydroxy- | 9.8 | 169.1 | 23.4 | 25.1 | 11 |
| Trioxocane | <LOQ | <LOQ | <LOQ | <LOQ | <LOQ |
| Sum | 283.8 | 2679.2 | 353.1 | 462.4 | 247.8 |
| Removal ratio (%) | | | 86.82 | 82.74 | 90.75 |
| FA (ug/m³) | | 1448 | 128 | 329 | 145 |
| AA (ug/m³) | | 15934 | 2825 | 2982 | 2187 |
| PA (ug/m³) | | 57.0 | 18 | 22 | 13 |
| acrolein (ug/m³) | | 2.0 | 3 | 12 | 3 |
| Sum | | 17441 | 2974 | 3345 | 2348 |
| DMD | | 0.478 | 0.060 | 0.078 | 0.040 |
| EMD | | 0.084 | 0.064 | 0.051 | 0.045 |
| Trioxocane | | 0.083 | 0.033 | 0.023 | 0.027 |
| Sum | | 0.645 | 0.157 | 0.152 | 0.112 |

Note:
units are in PPM (by way of the headspace GC-MS method) and LOQ is around 0.1 ppm (very low level); units are in μg/m³ (by way of SPME on-fiber derivatization method) and LOQ is around 1.0 μg/m³; units are in PPM (by ways of SPME (solid phase micro-extraction) GC-MS method) and LOQ is 0.005 ppm. DMD/EMD/Trioxocane are cyclic ethers that are usually formed as a derivation of propylene glycol and acetaldehyde or propionaldehyde during the storage.

TABLE 8
Odorant results of DPnB after 3-month heat-ageing at 54° C.

| | Examples | | | | |
|---|---|---|---|---|---|
| | | | | | IE-B16 |
| | CE-B12 | CE-B13 | IE-B14 | CE-B15 | AEEA + SVE |
| | Ref. | Ref. | AEEA | SVE | 100 ppm + |
| Additive | Fresh | Aged | 100 ppm | 100 ppm | 100 ppm |
| Butanal | 296.3 | 842.5 | 161.4 | 52.8 | 50.0 |
| Formic Acid, Butyl Ester | 439.4 | 1281.5 | 174.7 | 93.7 | 29.5 |
| Acetic Acid, Butyl Ester | 33.1 | 107.1 | 12.3 | 5.2 | 7.1 |
| 2-Propanone, 1-Hydroxy- | 30.7 | 136.7 | 15.4 | 1.1 | <LOQ |
| Propanoic Acid, 2,2-Dimethyl | 165.3 | 649.2 | 79.7 | 18.3 | 36.5 |
| Acetic Acid | 132.1 | 383.6 | 53.2 | 18.1 | 10.2 |
| 2-Propanone, 1-(Acetyloxy)- | 85.3 | 178.3 | 34.5 | 16.0 | 14.6 |
| 1,2-Propanediol, 1-Acetate | 88.1 | 282.3 | 24.9 | 12.4 | 2.4 |
| Sum | 1270.3 | 3861.2 | 556.1 | 217.6 | 150.3 |
| Removal ratio (%) | | | 85.60 | 94.36 | 96.11 |
| FA (ug/m³) | | 688.7 | 266 | 380 | 168 |
| AA (ug/m³) | | 14291.0 | 7922 | 12829 | 5173 |
| PA (ug/m³) | | 323.7 | 253 | 656 | 358 |
| acrolein (ug/m³) | | 2.3 | 22 | 69 | 11 |
| Sum | | 15305.7 | 8463 | 13934 | 5710 |
| DMD | | 0.768 | 0.242 | 0.404 | 0.224 |
| EMD | | 0.048 | 0.049 | 0.037 | 0.027 |
| Trioxocane | | 0.024 | 0.072 | 0.070 | 0.072 |
| Sum | | 0.840 | 0.363 | 0.511 | 0.323 |

Note:
units are in PPM (by way of the headspace GC-MS method) and LOQ is around 0.1 ppm (very low level); units are in μg/m³ (by way of SPME on-fiber derivatization method) and LOQ is around 1.0 μg/m³; units are in PPM (by ways of SPME (solid phase micro-extraction) GC-MS method) and LOQ is 0.005 ppm. DMD/EMD/Trioxocane are cyclic ethers that are usually formed as a derivation of propylene glycol and acetaldehyde or propionaldehyde during the storage.

TABLE 9
Odorant removal in Butyl CARBOTOL ™ Solvent

| | Examples | | | |
|---|---|---|---|---|
| | CE-BC1 | IE-BC2 | CE-BC3 | IE-BC4 |
| Odorant | Ref. | AEEA | SVE | AEEA + SVE |
| Butanal | 705.1 | 89.1 | 70.1 | 70.4 |
| Formic acid, Butyl ester | 791.8 | 38.6 | 39.3 | 24.1 |
| 1-Butanol | 359.7 | 58.1 | 56.0 | 56.6 |
| Ethanol, 2-methoxy- | 17.5 | <LOQ | <LOQ | <LOQ |
| 1-Propanol, 2,2-Dimethyl- | 227.3 | 23.5 | 20.5 | 19.9 |
| 1-Propanol | 17.5 | 0.4 | 0.1 | < LOQ |
| 1-Butanol, 2-Methyl- | 11.6 | 0.3 | <LOQ | <LOQ |
| Butanoic acid, 2-oxo- | 38.3 | 3.0 | 2.0 | 0.2 |
| Hydrocarbon ether | 24.0 | 0.1 | 0.6 | 0.1 |
| Ethanol, 2-Butoxy- | 499.3 | 258.2 | 265.0 | 243.4 |
| Isomer of ethanol, 2-butoxy | 418.7 | 25.9 | 29.5 | 14.7 |
| Ethylene oxide | 162.6 | 2.3 | 6.1 | <LOQ |
| 1,2-Ethanediol, Diformate | 191.3 | 9.2 | 17.3 | 5.8 |
| 2-Propanol, 1-(2-butoxyethoxy)- | 78.7 | 14.2 | 18.5 | 7.0 |
| Oxirane, (Butoxymethyl)- | 15.8 | 25.4 | 31.2 | 8.6 |
| 1,3-Dioxol-2-one | 72.3 | 8.7 | 6.9 | 3.8 |
| Hydrocarbon ether2 | 76.2 | 2.4 | 7.9 | 0.9 |
| Sum | 3707.7 | 559.4 | 571.0 | 455.5 |
| Removal ratio (%) | | 84.91 | 84.60 | 87.71 |

Note:
the units are in PPM ((by way of the headspace GC-MS method).

### III. Discoloration Test

Testing Methodology

In the series of experiments, around 20 mL of neat DOWANOL™ DPM without additives is stored at room temperature as Comparative Example 1 (CE-M17). A DPM sample without additives is stored at 54° C. as Comparative Example 2 (CE-M18). The DPM samples with DEA, AEEA or Tris-Amine are marked as Examples (IE-M19, IE-M20 or IE-M21). Samples containing SVE or PG are marked as CE-M22 and CE-M3 respectively. Samples with DOWANOL™ DPnB and the additives discussed above are then also prepared and labeled in the same manner but labeled with “B” instead of “M” in their names (see Table 10). Table 10 lists all the tested formulations.

To monitor the color evolution of the samples in the presence of the various additives, the color data is then measured. The color measurement is carried out with the color tester Ultra Scan VIS USVIS 2052. For each sample measurement, the sample cell is cleaned with deionized water and ethanol and dried by blowing with compressed air. Then, around 15 mL of solvent is poured into a test cell and put in the testing machine to compare with the reference sample. Results for tests conducted in this manner are listed below in Tables 11A and 11B.

TABLE 10
Discoloration Test Formulations

| Examples | Additive | Amount | Solvent |
|---|---|---|---|
| CE-M17 | Blank (RT¹) | N/A | DPM |
| CE-M18 | Blank (oven) | N/A | DPM |
| IE-M19 | DEA | 250 PPM | DPM |
| IE-M20 | AEEA | 250 PPM | DPM |
| IE-M21 | Tris-Amine | 250 PPM | DPM |
| CE-M22 | SVE | 250 PPM | DPM |
| CE-M23 | PG | 250 PPM | DPM |
| CE-B17 | Blank (RT1) | N/A | DPnB |
| CE-B18 | Blank (oven) | N/A | DPnB |
| IE-B19 | DEA | 250 PPM | DPnB |
| IE-B20 | AEEA | 250 PPM | DPnB |
| IE-B21 | Tris-Amine | 250 PPM | DPnB |
| CE-B22 | SVE | 250 PPM | DPnB |
| CE-B23 | PG | 250 PPM | DPnB |

Results

TABLE 11A
DPM Discoloration Test Results

| | | | After 6 | After 2 | After 4 | After 7 | After 10 | After 13 |
|---|---|---|---|---|---|---|---|---|
| Ex. | Additive | Initial | days | weeks | weeks | weeks | weeks | weeks |
| CE-M17 | Blank (RT) | 2.68 | 2.47 | 2.75 | 2.80 | 2.79 | 2.25 | 2.82 |
| CE-M18 | Blank (oven) | 2.52 | 2.37 | 2.37 | 2.84 | 2.80 | 2.62 | 2.75 |
| IE-M19 | DEA 250 ppm | 2.78 | 2.95 | 4.43 | 10.53 | 4.76 | 3.44 | 4.39 |
| IE-M20 | AEEA 250 ppm | 2.84 | 2.98 | 3.49 | 4.87 | 9.27 | 20.71 | 13.61 |
| IE-M21 | Tris-Amine | 2.75 | 2.20 | 2.30 | 2.91 | 3.04 | 2.22 | 3.26 |
| | 250 ppm | | | | | | | |
| CE-M22 | SVE 250 ppm | 3.40 | 3.98 | 4.73 | 5.85 | 6.91 | 6.45 | 8.26 |
| CE-M23 | PG 250 ppm | 2.77 | 9.39 | 35.91 | 68.93 | 105.94 | 128.75 | 151.14 |

Note:
the color units are Pt—Co.

TABLE 11B
DPnB Discoloration Test Results

| | | | After 6 | After 2 | After 4 | After 7 | After 10 | After 13 |
|---|---|---|---|---|---|---|---|---|
| Ex. | Additive | Initial | days | weeks | weeks | weeks | weeks | weeks |
| CE-B17 | Blank (RT) | 2.25 | 2.92 | 2.75 | 2.74 | 2.57 | 2.74 | 3.06 |
| CE-B18 | Blank (oven) | 2.25 | 2.73 | 2.97 | 2.83 | 2.25 | 2.79 | 2.14 |
| IE-B19 | DEA 250 ppm | 2.25 | 2.77 | 4.95 | 9.06 | 6.76 | 4.35 | 3.67 |
| IE-B20 | AEEA 250 ppm | 2.25 | 2.94 | 3.46 | 3.19 | 3.57 | 4.46 | 5.23 |
| IE-B21 | Tris-Amine | 2.25 | 2.71 | 2.71 | 2.75 | 3.67 | 4.72 | 4.60 |
| | 250 ppm | | | | | | | |
| CE-B22 | SVE 250 ppm | 2.85 | 4.66 | 5.14 | 6.22 | 6.51 | 7.98 | 9.16 |
| CE-B23 | PG 250 ppm | 2.25 | 10.82 | 21.48 | 38.98 | 60.25 | 75.24 | 83.89 |

Note:
the color units are Pt—Co.

### IV. Discussion

Based on the odorant removal results in DOWANOL™ DPM (Table 3) and DOWANOL™ DPnB (Table 4) DEA (IE-M3 and IE-B3) and AEEA (IE-M4 and IE-B4) performed surprisingly well in the removal of odorant impurities as compared to the blank control comparative example (CE-M1 and CE-B1).

Based on the analytical results of Headspace GC-MS in Table 5 and Table 6, the amino alcohols alone and combinations of amino alcohol and antioxidant demonstrated an unexpected improvement of odorant removal efficiency (IE-M8, IE-M9, IE-M10, IE-M11, IE-B8, IE-B9, IE-B10, IE-B11). One notable example being DEA and SVE in DPM, which showed a strong synergistic effect (IE-M11) between the amino alcohol and antioxidant.

Based on the analytical results of aged samples in Table 7 and Table 8, after the storage for 3 months at 54° C., the odorant impurity content increased significantly for both DPM and DPnB. Remarkably, AEEA (IE-M14 and IE-B14) maintained a very low amount of aldehyde content in DPM and DPnB after 13 weeks. The synergistic improvement of combining AEEA with an antioxidant was also observed in these tests (IE-M16 and IE-B16). Additionally, strong cyclic ether (DMD/EMD/Trioxocane) removal performance was observed for these examples.

Based on the data in Table 9 amino alcohol or the blend of amino alcohol and antioxidant work with EO based solvents (IE-BC2 and IE-BC4).

Based on the color stability results of additives in DOWANOL™ DPM (Table 11A) and DOWANOL™ DPnB (Table 11B) after the ageing at 54° C. for 13 weeks, the DPM and DPnB samples with additive didn't show a significant color change (IE-M19— CE-M22 and IE-B19-CEB22) except for the samples containing propyl gallate (CE-M23 and CE-B23).

1. An odor removal composition for oxygenated solvents comprising at least one alcohol amine with the structure of: 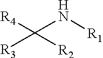
wherein, R₁, R₂ and R₃ are H, alkylamine, or hydroxyl alkyl group with linear or branched carbon chain ranging from C1-C8, and R₄ is an alkylamine or hydroxyl alkyl group with linear or branched carbon chain ranging from C1-C8.

2. The odor removal composition for oxygenated solvents of claim 1, wherein the composition further includes at least one antioxidant.

3. The odor removal composition for oxygenated solvents of claim 2, wherein the at least one antioxidant is a phenolic antioxidant.

4. The odor removal composition for oxygenated solvents of claim 1, wherein the at least one alcohol amine is aminoethyl ethanolamine, diethanolamine, or tris-(hydroxyl-methyl) amino-methane.

5. A method of controlling odor in oxygenated solvents by use of an odor removal composition, wherein the odor removal composition comprises at least an alcohol amine that has the structure of: 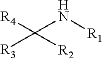
wherein, R₁, R₂ and R₃ are H, alkylamine, or hydroxyl alkyl group with linear or branched carbon chain ranging from C1-C8, and R₄ is an alkylamine or hydroxyl alkyl group with linear or branched carbon chain ranging from C1-C8.

6. The method of claim 5, wherein the composition further includes at least one antioxidant.

7. The method of claim 6, wherein at least one antioxidant is a phenolic antioxidant.

8. The method of claim 5, wherein the alcohol amine is aminoethyl ethanolamine, diethanolamine, or tris-(hydroxyl-methyl) amino-methane.

9. The method of claim 5, wherein the method is used to control the odor of one or more oxygenated solvents.

10. The method of claim 8, wherein the oxygenated solvent has the structure of R₁—O—(CHR₂CHR₃)O)nR₄, wherein R₁ ranges from C1-C9 linear or branched alkyl group or is a phenyl group, R₂ and R₃ are H or C1-C2 alkyl, wherein R₂ is H when R₃ is C1-C2, and R₃ is H when R₂ is C1-C2, and R₄ is H and n is an integer from 1-6.
